(19) United States
(12) Patent Application Publication
(10) Pub. No.: US 2010/0130829 A1
(43) Pub. Date: May 27, 2010

(54) MATERIALS AND METHODS FOR DETECTING EARLY CELL MIGRATION EVENTS

(76) Inventors: Ye Fang, Painted Post, NY (US); Vasiliy N. Goral, Painted Post, NY (US)

Correspondence Address: CORNING INCORPORATED, SP-TI-3-1, CORNING, NY, 14831, US

(21) Appl. No.: 12/277,579

(22) Filed: Nov. 25, 2008

(51) Int. Cl. A61B 5/00

(52) U.S. Cl. 600/300

(57) ABSTRACT

Aspects relate to the field of label-free biosensors, including optical biosensors and electric biosensors, for studying cellular behaviour particularly early events in cell migration, under a concentration gradient of stimulus. Various embodiments include devices and methods that enable the generation of a concentration gradient of stimulus for cells contacting with the surface of a biosensor including a label free biosensor. Some aspects also disclose methods to detect cellular responses upon such concentration gradient of a stimulus, as well as methods to detect the potency and efficacy of a stimulus using a single biosensor.

### FIELD OF THE INVENTION

Various aspects and embodiment relate to devices and methods for using label-free biosensors and flow chambers to monitor and/or assess the tendency of cells to migrate.

### BACKGROUND

Directed cell migration accompanies us from conception to death. For instance, this integrated process choreographs the morphogenesis of the embryo during its development and it plays a key role in metastasism of cancer cells. In developing embryos, failure of cells to migrate, or the migration of cells to inappropriate locations, can result in life threatening consequences such as the development of congenital brain defects. In adults, cell migration is central to homeostatic processes such as mounting an effective immune response and the repair of injured tissues. Furthermore, cell migration can contribute to pathologies including vascular disease, chronic inflammatory diseases, tumor formation and metastasis. Understanding the mechanisms underlying cell migration is also important in emerging areas of biotechnology that focuses on cellular transplantation and the manufacture of artificial tissues, as well as for the development of new therapeutic strategies for controlling invasive tumor cells.

Given the importance of cell migration events in human and animal health there is an on-going need for new devices and methods for studying this process. Aspects and embodiments disclosed herein address various devices and methods that can be used to detect, study, and measure cellular migration.

### SUMMARY

One aspect of the invention is a method for studying cell motility comprising the steps of: providing a device, which includes a biosensor; attaching cells to the bottom of the chamber; introducing at least one solution that includes at least one compound into the chamber through the camber inlet so as to form a compound gradient across the chamber; and monitoring the biosensor so as to detect changes in the cells in response to the compound gradient. In one embodiment the chamber includes an inlet for the admission of at least one solution, a top and a bottom. In one embodiment the chamber includes a top and a bottom, the bottom of the chamber may be formed by a biosensor or it may be adjacent to a biosensor. In one embodiment cells are attached either directly to the biosensor or adjacent to the biosensor. The bottom of the chamber includes or is adjacent to the biosensor. In one embodiment the chamber further includes at least one outlet. In one embodiment the biosensor is one of the following types of biosensors: a surface plasmon resonance sensor, a resonant waveguide grating sensor or an interferometer sensor.

Another aspect of the invention is a method for studying cell motility that further includes the steps of: collecting a first set of data at a first time point from the biosensor at, at least two distinct areas on the biosensor; gathering a second set of data at a second time point from the biosensor at, at least two distinct areas on the biosensor; and comparing the first and the second data sets for each location, and the cellular profiles at, at least two distinct areas on the biosensor.

In one embodiment the device used in a method for studying cell motility further includes a reservoir that stores at least one solution before it is introduced in to the chamber. The reservoir includes at least one outlet that may be connected either directly or indirectly to an inlet on the chamber. In another embodiment the device further includes a valve. The valve may include an inlet and an outlet; the valve outlet may be connected either directly or indirectly to the chamber inlet and the valve outlet may be connected either directly or indirectly to the reservoir outlet. In still another embodiment the device further includes a pump. The pump includes a pump inlet and a pump outlet in which the pump inlet may be connected either directly or indirectly to either a solution reservoir or to a valve and the pump outlet may be connected either directly or indirectly to ether a valve inlet or to the chamber inlet.

In still another aspect of the invention the method for studying cell motility includes providing a device that comprises a chamber with a first inlet for the introduction of a first solution into the chamber and a second inlet for the introduction of a second solution into the chamber in which the two solutions have different compositions. The solutions maybe introduced into the chamber such that a gradient of at least one compound in at least one of the solutions is formed in the chamber in the direction perpendicular to the flow across the cells on the bottom of the chamber. In one embodiment the solution is introduced into the chamber through the chamber inlet at a controlled flow rate and duration of time. In one embodiment the compounds in the solution includes at least one compound selected from the group including proteins, nucleic acids, fatty acids, cells viruses, small molecules, buffers, salts, detergents and the like.

In still another embodiment the method for studying cell motility includes a device that includes a temperature controller and/or sensor for regulating the temperature of at least one of the solutions introduced into the chamber.

Another aspect of the invention is a method for studying cell motility comprising the steps of: providing a device, that includes a biosensor and a chamber having at least one chamber inlet for admission of at least one solution, a top and a bottom, wherein the bottom of the chamber includes or is adjacent to the biosensor; attaching cells to the bottom of the chamber wherein the cells are in communication with the biosensor; introducing at least one solution, the solution including at least one compound, into the chamber through the chamber inlet such that there is a compound gradient formed across the chamber; monitoring through the biosensor and at least two locations, a cellular response imparted by the compound; and comparing the cellular responses from the at least two locations.

Still another aspect of the invention is a device suitable for monitoring cell migration, comprising: a biosensor; and a chamber in which the chamber has at least one chamber inlet for admission of at least one solution that includes at least one compound into the chamber so as to form a gradient in the chamber. In at least one embodiment the chamber has, a top and a bottom, wherein the distance between the top and the bottom of the chamber is in the range of about 200 microns to about 2 mm, and wherein the bottom of the chamber includes or is adjacent to the biosensor and includes at least one cell attached to the bottom of the chamber. In one embodiment of the invention the distance between the top and the bottom of the chamber is in the range of about 300 microns to about 1 mm.

### BRIEF DESCRIPTION OF THE FIGURES

FIG. 1a shows a side view of biosensor-based cell assays for studying cellular responses to a chemical gradient used in some embodiments of the disclosure.

FIG. 1b shows the top view of biosensor-based cell assays for studying cellular responses to a chemical gradient used in some embodiments of the disclosure.

FIGS. 2a-2b show aspects of a RWG biosensor-based cell assay for studying cellular responses to stimulation with a chemical used, in some embodiments of the disclosure.

FIGS. 3a-3b show aspects of a RWG biosensor-based cell assay for studying cellular responses upon a chemoattractant chemical gradient used in some embodiments of the disclosure.

FIGS. 4a-4d show the biosensor system having a chamber suitable for forming a chemical gradient, and the exemplary fluorescence images of a fluorescent chemical gradient at different time points used in some embodiments of the disclosure.

FIG. 5 shows the results of fluorescence intensity distribution across the biosensor, measured at three different time points after a fluorescent chemical gradient is formed.

FIGS. 6a-6d show the biosensor system having a chamber suitable for forming a chemical gradient, and label-free images collected by a biosensor of a cell layer in response to the chemical gradient at different time points, in one embodiment of the disclosure.

FIGS. 7a-7c show the biosensor system having a chamber for forming a chemical gradient, the label-free image collected by a biosensor from a cell layer upon exposure to a chemical gradient at a specific time point, and the optical responses of cells at different locations across the biosensor, in one embodiment of this disclosure.

FIGS. 8a-8b show the real-time kinetic responses of cells located at different locations across the biosensor, upon exposure to a chemical gradient, in one embodiment of the disclosure.

FIGS. 9a-9b show the theoretically calculated dose-dependent responses of cells to a non-chemoattractant chemical gradient, based on the responses shown in FIG. 8, in one embodiment of this disclosure.

FIG. 10 shows the top view of biosensor-based cell assays suitable for studying cellular responses to a chemical gradient formed by laminar flows of two different solutions.

### DETAILED DESCRIPTION

Reference will now be made in detail to embodiments of the invention, examples of which are illustrated in the accompanying drawings. Whenever possible, the same reference numerals will be used throughout the drawings to refer to the same or like parts.

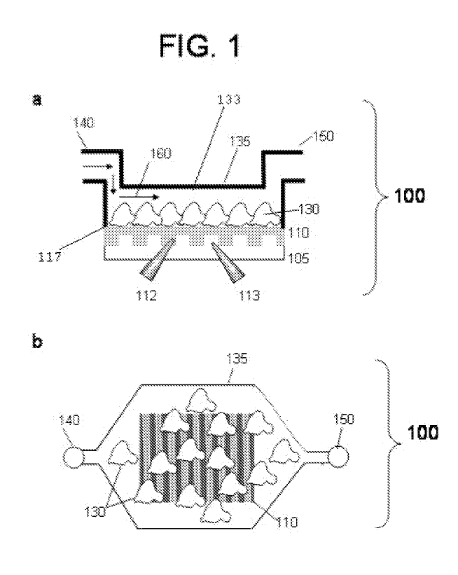

One aspect provides device for measuring cell migration as illustrated in FIG. 1. One embodiment, comprises a biosensor having a surface for attaching cells; a chamber for accommodating a solution, the chamber including a chamber inlet for introducing a solution into the chamber and (optionally) a chamber outlet for expelling the solution from the chamber, wherein the biosensor is positioned at the bottom of the chamber; and a detector that collects data from said biosensor. In various embodiments the biosensor may be selected from the group including a surface plasmon resonance sensor, a resonant waveguide grating sensor and an interferometer sensor. The biosensor in the device has a surface that is comprised of individual areas and the detector collects data from at least some of the individual areas. In a preferred embodiment, the device comprises an array of biosensors, wherein each biosensor is in communication with a separate chamber. The arrayed biosensor-chambers may be comprised of a series of wells, and the dimensions of this device may conform to standard SBS microtiter plate footprint. Such systems permit high throughput and high content label-free analysis of cell migration events. The solution includes at least one compound, and is introduced into the chamber such that it forms a gradient across the chamber.

In one embodiment of the device the detector collects data from the same individual areas of the biosensor at different times. These data may be processed to track changes in cells across the surface of the biosensor at different areas and at different times and in response to the introduction of different molecule, cell or solutions or gradients of the same of different temperatures and viscosities of various solutions.

In another embodiment the device includes a reservoir which stores and provides a solution to a pump that in turn supplies the solution to the inlet side of the chamber. In still another embodiment a valve may be positioned between the reservoir and chamber in either the presence of absence of a pump in the device. The pump, value or other means may be used to control the rate that the solution is introduced into or allowed to exit the chamber thereby changing the time that cells on the surface of the device remain in contact with the solution and its components, or to control the amount (i.e., volume) of the solution introduced into the chamber creating a chemical concentration gradient across the surface of the platform.

In an alternative embodiment the device includes at least two inlets which allow at least two different solutions to be introduced in parallel into the chamber independently at a controlled flow rate. Laminar flows of multiple different solutions can produce stable concentration gradients across the chamber. Varying the solutions flow rates can create a concentration gradient with a controlled gradient profile, wherein the gradient is perpendicular to the direction of the flow.

Another aspect is a method for measuring cell motility, comprising the steps of providing a device, also referred to as a platform, for measuring cell motility, said platform comprising: a biosensor, and a chamber, where the chamber includes at least one chamber inlet for introducing a solution into the chamber and optionally a chamber outlet for expelling the solution from the chamber. The biosensor may form the bottom of the chamber or it is positioned adjacent to the bottom of the chamber. At least one cell is attached to the bottom of the chamber. The device further includes a detector that collects data from at least two distinct areas of the biosensor or at least two distinct biosensors. The method further includes the steps of: contacting the cells on the surface with the solution introduced into the chamber through the chamber inlet, where the solution includes at least one compound and forms a concentration gradient across the surface; and monitoring changes in data collected by the detector at the least two at distinct areas on the biosensor (or two biosensors) at least two different times. In various embodiments the biosensor may be selected from the group including: surface plasmon resonance sensors, resonant waveguide grating sensors and interferometer sensors.

In one embodiment the chamber includes a top and a bottom and the distance between the top and the bottom of the chamber is on the order of between about 300 microns to about 1 mm. In still another embodiment the distance between the top of the chamber and the bottom of the chamber, which is the biosensor or is adjacent to the biosensor, is on the order of between about 200 microns to about 2 mm. A chamber with these dimensions is suitable for the label free detection of gradient induced changes in cell motility induced in the absence of laminar flow or at rates of laminar flow so low as to be substantially undetectable. In order to detect changes under these conditions it may also be necessary to adjust solution flow rates such that a gradient is formed and at least one cell motility event is detectable.

In one embodiment the method for measuring the effect of various compounds including, for example, proteins, nuclei acids, fatty acids, and small molecules, or various biological systems including, for example, viruses and cells or cellular migration. These methods may include the steps of collecting a first set of data from the biosensor at least two distinct areas on the biosensor at a first time point; adding at least one compound to the solution and introducing said solution including the at least one compound into the chamber such that the component forms a concentration gradient across the surface; and gathering a second set of data from the biosensor at least two distinct areas on the biosensor at a second time point. Data collected from cells attached to different areas of the surface in communication with the biosensor may change with changes in the chemical gradient across the surface as a solution introduced into the chamber travels towards the chamber outlet. Accordingly, data collected at times that correspond to changes in the chemical gradient of the solution introduced into the chamber can be processed to measure the concentration dependent effect of various components of the solution on cellular migration events.

Effective cell migration requires the seamless integration of localized, transient signaling events with changes in cellular architecture. Migration is a cyclical process in which a cell extends protrusions at its front and retracts its trailing end. It is often spurred into action by migration-promoting or chemotactic agents that induce an initial polarization.

In a quiescent non-migrating cell, adhesive forces maintain the cell's attachment to neighboring cells and the extracellular matrix. Upon encountering chemotactic molecules, cells may re-organize into a predominantly ‘front’, forward moving portion and a predominantly ‘back’, retracting rear portion that are defined by distinct signaling events; this process is known as polarization. In eukaryotic cells, cellular components such as Cdc42, PAR proteins (PAR6 and PAR3) and atypical protein kinase are thought to be involved in the initial polarization process. Changes in cellular architecture, such as the re-organization of the microtubule-organizing centre, microtubules and Golgi apparatus attachment to the front of the nucleus, appear to accompany polarization. The production of phosphatidylinositol trisphosphate (PIP₃) at the leading edge by the action of phosphoinositide 3-kinase (PI3K) has also been implicated in polarization. PTEN, a PIP3 phosphatase, localized to the rear and sides of the cell, helps to regulate the levels of PIP₃. Once polarization is initiated it is maintained by a set of overlapping positive feedback loops involving for example, PI3K, microtubules, Rho family GTPases, integrins and vesicular transport.

The process of protrusion marks the start of the migration cycle. The actin cytoskeleton provides the basic machinery for protrusion. Actin filaments themselves are intrinsically polarized; fast-growing “barbed” ends and slow-growing “pointed” ends provide an inherent drive for membrane protrusion. This can take the form of spike-like filopodia, in which actin filaments form long parallel bundles, or large, broad lamellipodia, where actin filaments form a branching ‘dendritic’ network. These two forms of protrusion appear to have very distinct functions. The parallel bundle design of filopodia means they are well suited to act as sensors and explore the local environment, whereas the branched design of lamellipodia forms broad protrusions in the direction of migration, providing a strong foundation over which the cell can move forward.

Actin filaments in filopodia elongate at their barbed ends and disassemble releasing actin monomers at their pointed ends, thus, a filament tread-milling mechanism rather than a branched nucleation mechanism is believed to be responsible for filopodial protrusion. The enrichment of proteins such as Ena/VASP, which binds barbed ends and antagonizes capping and branching, and the bundling protein fascin at filopodial tips greatly increases the ability of filopodia to push the plasma membrane forward.

In lamellipodial protrusions a branched nucleation mechanism forms a wide net-like structure which pushes the plasma membrane forward. The Arp2/3 complex induces the formation of new actin filaments by binding to the sides or tip of pre-existing ‘mother’ filaments. The Arp2/3 complex is activated locally at the cell membrane by Wasp/Wave family members which are themselves major targets for Cdc42 and Rac.

The rate and organization of actin polymerization in protrusions is regulated by several actin-binding proteins that affect the pool of available actin monomers and free ends. Profilin binds and targets actin monomers to barbed ends thereby preventing self-nucleation. Capping proteins terminate elongation and limit polymerization to new filaments at the plasma membrane, whereas proteins of the ADF/cofilin family sever filaments and promote actin dissociation from the pointed end. Within the dendritic network of lamellipodial structures other proteins such as cortactin, filamin A and actinin help to stabilize branches and cross-link actin filaments.

For a cell to advance, newly extended protrusions must attach to the surroundings and stabilize, providing a means of traction for the cell to pull itself forward. The physical component of traction is provided by the action of integrins in adhesions. Tractional force is created at sites of adhesion by the contractile properties of myosin II interacting with actin filaments attached indirectly to integrins at these adhesion sites.

Integrins are a major family of migration-promoting receptors that enable the outer surface of a protruding cell to adhere to the surface over which it is moving. By linking via adaptor proteins to actin filaments, integrins facilitate the cell to pull the bulk of its body forward. Integrins are heterodimeric receptors with a short domain projecting into the cytoplasm and a large extracellular domain. They act as both traction sites and mechanosensors transmitting information about the physical state of the surface over which the cell is moving and enabling it to make the necessary cytoskeletal adjustments. Ligand binding induces conformational changes that are transmitted to the cytoplasmic domains, integrin clustering and the activation of intracellular signaling cascades that lead to changes in the rate of phosphoinositol lipid synthesis, protein phosphorylation and activation of small GTPases. These signaling pathways are involved in establishing and maintaining cell polarity, regulating the formation and strength of adhesions as well as in modulating the organization and dynamics of the cytoskeleton. Activated integrins localize to the leading edge of migrating cells and are associated with the formation of new adhesions. While PKC or the GTPase, Rap1 act through talin to promote integrin activation and increase integrin affinity, leading to adhesion assembly, the kinase Raf-1 suppresses integrin activation and favors disassembly.

The turnover of adhesions is critical for effective migration. To extend a protrusion, the adhesions in that portion of the cell must disassemble. Once the protrusion has been extended, adhesions are re-assembled to provide the required traction for the cell to pull forward. At the same time, disassembly of adhesions at the cell rear is necessary to enable forward progress. The mechanisms of adhesion assembly and disassembly are still rather poorly understood, but clearly require tight spatial and temporal regulation. The initial clustering of integrins, stimulated by the multivalent nature of the extracellular matrix to which the cell is adhering, is thought to initiate adhesion assembly, but the types of adhesions which form vary greatly depending on the cell type and pliability of the substratum. Rapidly migrating blood cells, such as leukocytes for example, display few integrin clusters and their submicroscopic adhesions facilitate rapid movement. Normally non-migratory or slow moving cells display focal adhesions, large integrin clusters that are tightly adherent and appear to be dependent on Rho-stimulated myosin contractility. Small adhesions, also known as focal complexes, are commonly observed at the leading edge of migrating cells and have been shown to be dependent on Rac and Cdc42. These types of adhesions tend to stabilize lamellipodia and contribute to efficient migration.

The disassembly of adhesions at the rear of the cell and the retraction of the cell's tail complete the cycle of migration and enable cell translocation. Myosin II is crucial for retraction and the development of tension between adhesions at the rear and the retraction machinery. This tension can be sufficient to open stretch-activated calcium channels and lead to the activation of calpain. This protease contributes to adhesion disassembly at the cell rear by cleaving a number of focal adhesion proteins, including integrins, talin, vinculin and FAK. Disruption of PAKa and Rho/Rho kinase signaling, which regulates myosin II, severely impairs retraction in migrating cells. The release of adhesions at the rear of the cell facilitates protrusive activity at the front of the cell, contributes to the overall polarization and provides positive feedback for the continued cycle of migration.

Cell migration involves complex protein signaling cascades and changes in the structure of the cell's actinomyosin cytoskeleton. Although some cell movements are random, others are directed toward a signal. One such process, chemotaxis—which occurs in response to a chemical gradient—has been extensively studied. A classical way to study chemotaxis is to use a two-chamber system: cells are placed in one chamber and the chemical signal, or chemoattractant, in the other. Several variations of two chamber systems have been used including a top-and-bottom configuration known as the Boyden chamber, a side-by-side chamber configuration known as a Zigmond chamber and the Dunn chamber, which consists of concentric rings.

The Boyden chamber was likely the first two-chamber system to be described. It is still used today to study many types of cell migration, including chemotaxis, although the original design has been modified and improved upon since the system was first described in 1962. Separating the two chambers is a porous membrane through which cells can actively migrate. The size of the membrane pores is critical when using a Boyden chamber, and companies sell membranes with a variety of pore sizes. A typical eukaryotic cell is 30-50 microns in size and can fit through pores in the 3-13 micron size range, but various companies have optimized pore size for specific cell types. For example, Millipore supplies the QCM Quantitative Cell Migration assay that uses an 8 μM pore size membrane, optimal for epithelial and fibroblast cell migration assays.

Another classic method for estimating cellular migration, in the low-tech category, is the ‘wound healing’ assay, which is used to study migration of cells in the presence of a chemoattractant. A confluent plate of cells is ‘wounded’ by scraping a specific area of the plate. Migration of cells can then be monitored over time by imaging the cells as they move from the untouched areas into the wounded area. Although this assay is simple, it is also cumbersome because imaging cells over long periods can be difficult, and at first glance the system does not appear to be amenable to automation. Still, for example, Applied BioPhysics has developed a wound healing assay based on the concept of electric cell-substrate impedance sensing (ECIS). Briefly, one small electrode is placed on the bottom of a tissue culture well in the center, and a second electrode at the edge of the well completes the circuit. Cells that attach to the electrode in the culture well alter the current, and this effect can be measured directly. By growing the cells to confluence and then applying an electric field for 10-15 seconds, one kills the cells on the electrode. As the surrounding cells migrate into the wounded area, the associated change in current can be measured to calculate the cell migration rate.

Among the most popular combination of labeling method and microscopy system is speckle microscopy. Time lapse speckle microscopy is an excellent tool for studying the molecules involved in cell migration, but the technique does have drawbacks. Speckle microscopy is limited to processes that happen at a time scale of half a second or slower, while methods like fluorescent correlation spectrometry can detect binding of molecules at a millisecond time scale.

Various aspects and embodiments disclosed herein address the drawbacks of many of the currently available cell assays used to study cell migration.

Embodiments disclosed herein use label-free biosensor systems having high spatial resolution, in conjunction with various methods to generate a chemical gradient, and can be used to study cellular responses to compounds and conditions involved in cellular migration. Such responses can be used for determining the ability of a chemical or biochemical or biological to cause cell migration, as well as for determining the potency and efficacy of a chemical or biochemical or biological to cause cell signaling. Some aspects presented herein are particularly suited for studying early events in cell migration.

### 1. Label-Free Optical Biosensors and Biosensor-Based Cell Assays

Label-free cell-based assays generally employ a biosensor to monitor ligand-induced responses in living cells. A biosensor typically utilizes a transducer such as an optical, electrical, calorimetric, acoustic, magnetic, or like transducer, to convert a molecular recognition event or a ligand-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors that are applicable to the present invention include, but not limited to, optical biosensor systems such as surface plasmon resonance (SPR) and resonant waveguide grating biosensors and interferometer biosensors.

SPR and systems—SPR relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by, free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface.

RWG biosensors and systems—An RWG biosensor can include, for example, a substrate (e.g., glass) with an embedded grating structure, a waveguide thin film, and a cell layer. A photonic crystal biosensor is a RWG biosensor. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent in nature, meaning that it decays exponentially from the sensor surface; the distance at which it decays to 1/e of its initial value is known as the penetration depth and is a function of the design of a particular RWG biosensor, but is typically on the order of about 200 nm. This type of biosensor exploits such evanescent waves to characterize ligand-induced alterations of a cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured. The resonance conditions are influenced by the cell layer (e.g., cell confluency, adhesion and status), which is in direct contact with the surface of the biosensor. When a ligand or an analyte interacts with a cellular target (e.g., a GPCR, a kinase) in living cells, any change in local refractive index within the cell layer can be detected as a shift in resonant angle (or wavelength).

The Corning® Epic® system uses RWG biosensors for label-free biochemical or cell-based assays (Corning Inc., Corning, N.Y.). The Epic® System consists of an RWG plate reader and SBS (Society for Biomolecular Screening) standard sized microtiter plates having any number of wells, but typically either 96 or 384. Each well contains a biosensor in the well bottom. The detector system in the plate reader exploits integrated fiber optics to measure the shift in wavelength of the incident light, as a result of ligand-induced changes in the cells. A series of illumination-detection heads are arranged in a linear fashion, so that reflection spectra are collected simultaneously from each well within a column of a 384-well microplate. The whole plate is scanned so that each sensor can be addressed multiple times, and each column is addressed in sequence. The wavelengths of the incident light are collected and used for analysis. A temperature-controlling unit can be included in the instrument to minimize spurious shifts in the incident wavelength due to the temperature fluctuations. The measured response represents an averaged response of a population of cells. An onboard liquid handling device may also be integrated into the reader system such that multiple solutions can be simultaneously added into the wells of a biosensor microplate having cells. Alternatively, a liquid handling accessory device can be attached to the outside of the reader system.

Electrical biosensors and systems—Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance measurement for whole cell sensing was first realized in 1984. Since then, impedance-based measurements have been applied to study a wide range of cellular events, including cell adhesion and spreading, cell micromotion, cell morphological changes, and cell death. Classical impedance systems suffer from high assay variability due to use of a small detection electrode and a large reference electrode. To overcome this variability, the latest generation of systems, such as the CellKey system (MDS Sciex, South San Francisco, Calif.) and RT-CES (ACEA Biosciences Inc., San Diego, Calif.), utilize an integrated circuit having a microelectrode array.

Biosensor output signals of living cells upon stimulation is an integrated response—Cells are dynamic objects with relatively large dimensions, e.g., typically tens of microns. RWG biosensors enable detection of ligand-induced changes within the bottom portion of cells, determined by the penetration depth of the evanescent wave (i.e. to a range of approximately 200 nm). Furthermore, the spatial resolution of an optical biosensor is determined by the spot size (about 100 microns) of the incident light source. Thus, a highly confluent cell layer is generally used in order to achieve optimal assay results; and the sensor configuration can be viewed as a three-layer waveguide composite, including, for example, a substrate, waveguide thin film, and a cell layer. Following a 3-layer waveguide biosensor theory in combination with cellular biophysics, we found that for whole-cell sensing, a ligand-induced change in effective refractive index, the detected signal AN, is governed by equation (1):

$$\begin{matrix}Δ N = S ( N ) Δ {n}_{C} \\ = S ( N ) α d {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ]\end{matrix} \qquad ( 1 )$$

where S(C) is the system sensitivity to the cell layer, and Δn_{c} is the ligand-induced change in local refractive index of the cell layer sensed by the biosensor. ΔA_{c} is the penetration depth into the cell layer, α is the specific refractive index increment (about 0.18/mL/g for proteins), zᵢ is the distance where the mass redistribution occurs, and d is an imaginary thickness of a slice within the cell layer. Here the cell layer is divided into an equally-spaced slice in the vertical direction. We assumed that the detected signal is, to a first order, directly proportional to the change in refractive index of the bottom portion of cell layer Δn_{c}. The Δn_{c} in turn is directly proportional to changes in local concentration of cellular targets or molecular assemblies within the sensing volume, given the refractive index of a given volume within cells is largely determined by the concentration of biomolecules, mainly proteins. A weighted factor exp(−zᵢ/ΔZ_{c}) is taken into account for a change in local protein concentration that occurs, considering the exponentially decaying nature of the evanescent wave. Thus, the detected signal is the sum of mass redistribution occurring at distinct distances away from the sensor surface, each with unequal contribution to the overall response. Eq. (1) suggests that the detected signal with an RWG biosensor is sensitive primarily to the vertical mass redistribution, as a result of a change in local protein concentration. The detected signal is often referred to as a dynamic mass redistribution (DMR) signal.

In a typical impedance-based cell assay, cells are brought into contact with a gold electrode arrayed on the bottom of culture wells. The total impedance of the sensor system is determined primarily by the ion environment surrounding the biosensor. Under application of an electrical field, the ions undergo field-directed movement and concentration gradient-driven diffusion. For whole cell sensing, the total electrical impedance has four components: the resistance of the electrolyte solution; the impedance of the cell; the impedance at the electrode/solution interface; and the impedance at the electrode/cell interface. In addition, the impedance of a cell comprises two components: the resistance; and the reactance. The conductive characteristics of cellular ionic strength provide the resistive component, whereas the cell membranes, acting as imperfect capacitors, contribute a frequency-dependent reactive component. Thus, the total impedance is a function of many factors, including, for example, cell viability, cell confluency, cell numbers, cell morphology, degree of cell adhesion, ionic environment, the water content within the cells, the detection frequency, and like considerations. Therefore, a bioimpedance signal of living cells upon stimulation is also an integrated cellular response.

Because of its integrated nature, a biosensor output signal can be used to study cell signaling under different stimulation conditions.

High spatial resolution biosensor imaging systems—Optical biosensor imaging systems, including SPR imaging system, ellipsometry imaging, and RWG imaging system, offer high spatial resolution, and are preferably used in various embodiments. For example, SPR imager®II (GWC Technologies Inc) uses prism-coupled SPR, and takes SPR measurements at a fixed angle of incidence, and collects the reflected light with a CCD camera. Changes on the surface are recorded as reflectivity changes. Thus SPR imaging collects measurements for all elements of an array simultaneously.

Another approach involves a swept wavelength optical interrogation system based on RWG biosensor for imaging-based application. In this system, a fast tunable laser source is used to illuminate a sensor or an array of RWG biosensors in a microplate format. The sensor spectrum can be constructed by detecting the optical power reflected from the sensor as a function of time as the laser wavelength scans, and analysis of the measured data with computerized resonant wavelength interrogation modeling results in the construction of spatially resolved images of biosensors having immobilized receptors or a cell layer. Two-dimensional label-free images can be obtained without moving parts.

Alternatively, an angular interrogation system with transverse magnetic or p-polarized TM₀ mode can also be used. This system consists of a launch system for generating an array of light beams such that each illuminates a RWG sensor with a dimension of approximately 200 μm×3000 μm, and a CCD camera-based receive system for recording changes in the angles of the light beams reflected from these sensors. The arrayed light beams were obtained by means of a beam splitter in combination with diffractive optical lenses. This system allows up to 49 sensors (in a 7×7 well sensor array) to be simultaneously sampled at every 3 seconds.

### 2. Devices for Generating a Chemical Gradient

The present invention discloses devices for generating a chemical gradient and the label free detection of cell migration events in cells attached to the device. As shown in FIG. 1, the device (100) consists of an optical biosensor, and a chamber (133). The biosensor may form the bottom of the chamber (117) or lie adjacent to the bottom of the chamber 117. The chamber also includes a chamber top (135). The optical biosensor may be a surface plasmon resonance-based biosensor, or a resonant waveguide grating biosensor, or an optical interferometer-based biosensor. The bottom of the chamber (117) may be part of the biosensor or may lie adjacent to the plane of the biosensor. The chamber consists of an inlet (140) or optionally at least one additional inlet (not shown) and (optionally) an outlet (150). When a resonant waveguide grating biosensor is used as exampled in FIG. 1, in some embodiments, the biosensor can include, for example, a glass substrate (105) having a grating structure embedded therein, a waveguide (110) thin film, a light source (112), and a means to detect and process the resulting refracted light (113). Only the mass redistribution within the detection zone and the bottom portion of cells (130) is directly measured. Cells (130) can be directly cultured onto the surface of the biosensor, or brought to contact with the sensor surface. FIG. 1a shows the side view of a biosensor having a chamber for studying cellular responses upon exposure to a chemical gradient, whereas FIG. 1b shows the top view of a biosensor through the top of the chamber. In one embodiment the distance between the top (135) and the bottom (117) of the chamber (133) is on the order of between about 300 microns to about 1 mm. In still another embodiment the distance between the top (135) and the bottom (117) of the chamber (133) is on the order of between about 200 microns to about 2 mm.

The present invention discloses methods to form a chemical gradient across the whole biosensor. A solution of a defined volume containing a chemical or biochemical or biological or virus or cell is introduced into the chamber through the inlet (140), such that the introduced solution initially covers only a specific portion (160) of the chamber nearest to the inlet, but not the whole sensor area. The compound, e.g. chemical or biochemical or biological virus or cell is either freely dissolved in the solution, or encapsulated in a polymer, which is able to release the encapsulated molecules in a controlled manner. The concentration gradient is achieved by chamber-confined diffusion. As a result, the cells on the whole sensor area sense a gradient of the chemical or biochemical or biological virus or cell—the cells near the inlet see a high concentration of the added compound, whereas the cells near the outlet area see no or little added compound, due to the constrained diffusion of the stimulus molecules in the chamber. In one embodiment the compound stimulates cellular migration.

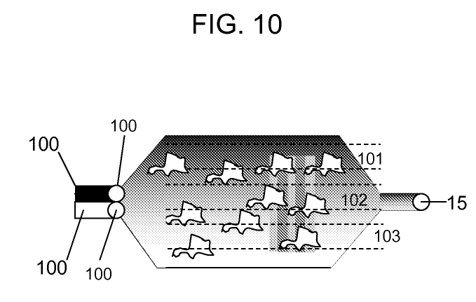

Alternatively, a chemical gradient can also be formed by flowing two independent solutions (e.g., a buffered solution, and a buffered solution containing a chemical at a given concentration) through two inlets of the chamber in parallel. Such parallel flow approach can result in the formation of a dynamic gradient across the whole biosensor in a direction perpendicular to the direction of flow. This is accomplished by diffusion in the direction perpendicular to the flow. As shown in FIG. 10, the chamber has two inlets (1001, and 1002) and an outlet (150). Two solutions (1003 and 1004) flow into the chamber through the inlet 1001 and 1002, respectively. As a result of diffusion of chemicals in the laminar flow, a concentration gradient of the chemical is formed in a direction perpendicular to the direction of flow. The biosensor can be divided into multiple zones that correspond to the concentration gradient direction for detecting changes in cells exposed to different concentrations of compounds in the flowing solutions, for example, zone 1010, 1020, and 1030.

Alternatively, a chemical gradient can also be formed by flowing two independent solutions (e.g., a buffered solution, and a buffered solution containing a chemical at a given concentration) through two inlets of the chamber in parallel. Such parallel flow approach can result in the formation of a dynamic gradient across the whole biosensor in a direction perpendicular to the direction of flow. This is accomplished by diffusion in the direction perpendicular to the flow. As shown in FIG. 10, the chamber has two inlets (1001, and 1002) and an outlet (150). Two solutions (1003 and 1004) flow into the chamber through the inlet 1001 and 1002, respectively. As a result of diffusion of chemicals in the laminar flow, a concentration gradient of the chemical is formed in a direction perpendicular to the direction of flow. The biosensor can be divided into multiple zones that correspond to the concentration gradient direction for detecting changes in cells exposed to different concentrations of compounds in the flowing solutions, for example, zone 1010, 1020, and 1030. In still another embodiment a gradient is formed within the chamber and cell migration events can be detected even in the absence of a the absence of laminar flow so long as the dimensions of the chamber are such that the distance between the top of the chamber (135) and the bottom of the chamber (117) are on the order of between about 300 microns to about 1 mm. And the flow rate of the solution forming the gradient is such that it produces a detectable change in the motility of the cell attached to the bottom of the chamber.

Alternatively, a scanning wavelength interrogation system can also be used. In this system, a polarized light covering a range of incident wavelengths with a constant angle is used to illuminate and scan across a waveguide grating biosensor, and the reflected light at each location can be recorded separately and used for determining location dependent cellular responses.

Alternatively, for the angular interrogation resonant waveguide grating biosensor system which utilizes a light band for illumination of the biosensor, this system measures cellular responses upon stimulation for cells located at a dimension across the whole biosensor within a specific narrow path (˜100 microns×2000 microns), leading to a resonant band (WO2006108183, Fang, Y., et al., Label-free Biosensors and Cells). The optical responses obtained represent an averaged response of a population of cells across the whole sensor in a narrowed band. The resulted resonant band can be subdivided into multiple areas; cells at each specific area within the light illuminating area can be examined.

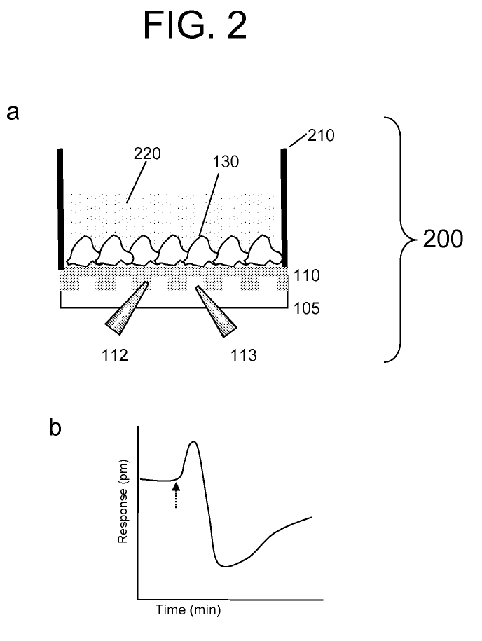

Referring to the figures, FIG. 2 shows a typical biosensor cell-based assay (200) for studying cell signaling. Here, an RWG biosensor is used for monitoring stimulation mediated dynamic mass redistribution (DMR) signal in living cells (130). The biosensor is located at the bottom of a well (210) in a microplate (such as 96 well SBS compatible microplate, or 384well SBS compatible microplate or 1536well SBS compatible microplate). A ligand (220) is introduced into the biosensor system by pipetting or using another liquid handling device (either onboard, or attached liquid handling accessory device), such that cellular receptors at any area of a cell see the same concentration of ligand molecules almost at the same time. This type of stimulation is termed a global stimulation. In this embodiment the biosensor has the cells (130) adhered onto the sensor surface (110). As a result of the global stimulation, the ligand added to the cells leads to an optical response of the living cells (FIG. 2b). The example shown here is epidermal growth factor (EGF)-induced optical response of quiescent A431 cells cultured measured using a RWG biosensor. The graph shows a time-dependent resonant wavelength shift in terms of picometers before and after the stimulation with EGF. A431 cells were directly cultured onto Corning 384well Epic® cell assay microplate. EGF of 32 nM was added into the cell medium solution (1×HBSS buffer, 1×Hank's balanced salt saline buffer, 20 mM Hepes-KOH, pH7.1) covering the quiescent A431 cells by an on-board liquid handler. The broken arrow indicated the time when EGF was introduced. The scanning wavelength interrogation Epic® system was used for this measurement. After simulation, the cellular response consists of an initial positive-dynamic mass redistribution (P-DMR) signal, followed by a slow decayed signal (negative-DMR, N-DMR) and a slowly increased signal (recovery P-DMR, RP-DMR). The P-DMR is largely due to the recruitment of intracellular proteins to the activated EGF receptors located at the basal membrane surface, whereas the N-DMR is largely due to the receptor internalization and cell detachment. The RP-DMR is largely due to the reattachment of cells to the sensor surface. The cytoskeletal remodeling plays important roles throughout the receptor signaling process.

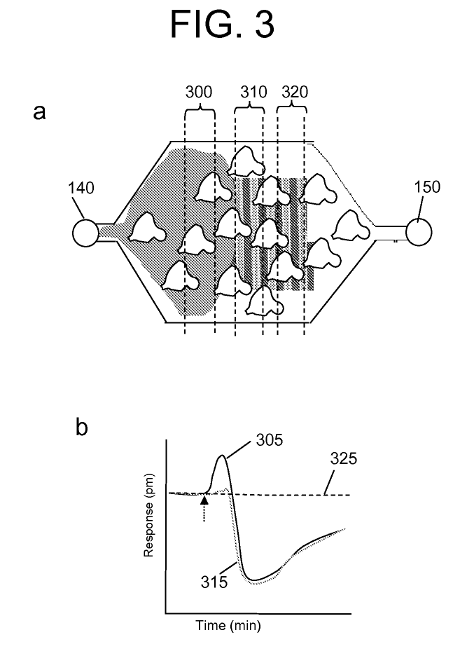

FIG. 3 shows an RWG biosensor for monitoring responses of cells located at different locations of a biosensor surface. Here the biosensor is located within a chamber comprising either the bottom of the chamber or laying adjacent to the bottom of the chamber. The chamber further includes a top portion. In some embodiments the distance between the top of the chamber and the bottom of the chamber is between about 300 microns to about 2 mm. In still another configuration the distance between the top and the bottom of the chamber is between about 200 microns and 2 mm. The chamber consists of an inlet (140) for introducing a chemical solution (350), and (optionally) an outlet (150) for expelling extra solution. In this example, the total volume of solution within the chamber is estimated to be about 6 microliters. Cells, such as A431 cells, are directly cultured or brought into contact with the surface of the biosensor within the chamber. A solution of a given volume (typically much smaller than the total volume of the chamber), such as a buffered solution containing EGF, is introduced into the chamber through the inlet by using a controlled pump or a pipetter. The solution added only reaches a portion of the chamber, such that only a portion of the biosensor surface immediately proximate to the inlet is exposed to the solution. The chemical or stimulus or other compound in the solution will diffuse away, thus forming a gradient. Examples shown are EGF gradient-triggered responses of quiescent A431 cells at the different locations, as indicated in FIG. 3a (the area 300, 310, and 320). When an EGF solution of 1.5 microliters was introduced into the chamber, the EGF solution added initially only reached the left side of the biosensor surface near the inlet area. The cellular responses at different locations (300, 310, and 320) were dramatically different, as shown in graphs of 305, 315 and 325, respectively. The cells at the area near the inlet (300) gave rise to a response (305) similar to that obtained using the global stimulation (FIG. 2b), since the cells sensed uniform concentrations of EGF from all directions. The cells at the area closer to the edge of the EGF solution (310) initially added sense an EGF concentration gradient, and responded with a truncated optical response, which lacked the initial P-DMR event (315), suggesting that there is no recruitment of intracellular proteins to the basal cell membranes. However, since the cells sense such EGF gradient, the cells tend to move towards these EGF molecules, leading to a response that is dominant by cell detachment—a critical process for cell migration. Conversely, the cells near the outlet area (320) were not exposed to EGF directly as those cells are far away from the concentration gradient, and thus those cells did not give rise to any obvious optical response (325).

Experimental Procedures

### Materials

EGF (epidermal growth factor) was obtained from Sigma Chemical Co. (St. Louis, Mo.). Tetramethylrhodamine labeled EGF (TMR-EGF) and Texas Red-labeled phalloidin (TR-phalloidin) were obtained from Molecular Probes (Eugene, Oreg.). Corning® Epic® 384 and 96-well biosensor inserts were obtained from Corning Inc. (Corning, N.Y.) and cleaned by exposure to high-intensity UV light (UVO cleaner, Jelight Co. Inc., Laguna Hills, Calif.) for 6 minutes before use. An Epic® 384well biosensor microplate cell culture compatible system was obtained from Corning Inc (Corning, N.Y.) and used directly without any pretreatment.

### Fabrication of PDMS Chambers and Assembly of Biosensor Device

The first step of fabrication consists in generating a silicon master. This was achieved by U.V. photolithography on a Si [100] wafer. Briefly, 4″ silicone wafers were primed with P-20 immediately before the resist was applied. 1 μm thick Shipley 1813 photoresist was spun on the wafer at 3000 rpm for 30 sec (acceleration 1000 rpm/s) and soft baked on a hot plate for 1 min at 110° C. The wafers were exposed to UV-light through a chromium mask with the desired structures designed as CAD-drawing using MA6 (Karl Zeiss) mask aligner. After a post bake period of 2 min at 80° C. the wafers were finally developed (60-100 s, MF-319, Shipley), thoroughly rinsed with water and dried. Molds for 200 μm deep fluidic channels and cell culture chambers were etched into the silicone using Plasma Therm 72 fluorine based reactive ion etcher. After photoresist stripping and cleaning silicone masters were exposed to trichloro(1H, 1H, 2H, 2H)-perfluorooctyl vapor for 2 h for pasivation.

PDMS replicas of structures were produced by pouring a PDMS precursor mixture over the whole 4″ silicon master (1:10 curing agent to prepolymer ratio, Sylgard 184, Dow Corning, US). It was then cured at 70° C. for 80 min. The cured PMDS was peeled of from the silicone mold to complete the fabrication. Similarly, a PDMS device having a array of 4×6 chambers can be fabricated using similar approach.

Once the PDMS chamber were made, these substrates were subject to surface oxidation using O₂ plasma cleaning for 30 sec at pressure of 500 mTorr, and attached onto the biosensor insert such that within a chamber there is a single biosensor locating within the center of the chamber. Afterwards, each chamber was filled with 75% ethanol twice, each 30 sec, followed by washing with PBS buffer and drying. Similarly, the array of 4×6 chamber PDMS device was attached to the 96well biosensor insert, such that within each chamber, there is a RWG biosensor located at the center of the chamber. The 96well biosensor insert consist of an array of 8×12 biosensors in a footprint that is compatible with a SBS 96 well microplate. Each chamber has an inlet, and an outlet. The biosensor dimension is about 2 mm×2 mm. Similarly a 384well biosensor insert can also be used.

### Cell Culture

Human epidermoid carcinoma A431 cells were obtained from American Type Cell Culture. A431 cells were grown in Dulbecco's modified Eagle's medium (DMEM) supplemented with 10% fetal bovine serum (FBS), 4.5 g/liter glucose, 2 mM glutamine, and antibiotics. For cell culture, ˜4×10⁴ cells suspended in 6 μl the medium containing 10% FBS were injected into the chamber, and were cultured at 37° C. under air/5% CO₂ until ˜95% confluency was reached (˜1 day). To prevent drying during the culture, the biosensor device was maintained within a petri dish with a cover, and extra cell medium within the dish. The cells were then subject to starvation with the medium without any FBS for overnight. Because the biological status of cells (e.g., cell viability, confluency and degree of adhesion) could significantly impact the measurement of mass redistribution, we inspected the quality of cells using both light microscopy and the optical system. The optical system allows one to collect the resonant image of whole sensor at the single cell.

Characterization of a Chemical Gradient within the Biosensor Device

To characterize the chemical gradient within the biosensor device, the biosensor device having confluent A431 cells was subject to injection of fluorescently labeled EGF (TMR-EGF of 32 nM), and subsequent fluorescent imaging with a Zeiss microscope at different time points.

### Label-Free Biosensor Cell Assays

Coming RWG imaging system was used for label-free biosensor cell assays. The imaging system is capable of high resolution imaging, with a spatial resolution of 6 microns in the direction perpendicular to the propagation path of the coupled light, a resolution of about 150 microns in the direction parallel to the propagation path of the coupled light. The cellular responses across the whole sensor were recorded in real time at each pixel (6 microns×6 microns). The cellular responses at defined locations or zones from the inlet to the outlet were averaged to generate location-dependent cellular responses.

The A431 cells reached high confluency before assays, and were subject to starvation for overnight with serum free medium. Before assays, the cell medium was replaced with 1×Hanks' balanced salt solution (HBSS) having 20 mM Hepes buffer and 0.1% bovine serum albumin (BSA), and incubated within the imaging system for about 1 hour. The chamber was filled with 6.6 micro-liters buffered solution. To generate a chemical gradient, 1.2 micro-liter EGF in 0.1% BSA HBSS solution was injected into the chamber from the inlet using a controlled pump. The cellular responses before and after EGF addition were recorded using the high resolution imaging system. Depending on the biosensor insert used, the biosensor has a dimension of 3 mm×3 mm or 2 mm×2 mm. The chamber dimension is a length of about 9 mm from the inlet to the outlet, a width of 5 mm, and a height of 200 microns from the sensor surface to the bottom surface of the chamber. The total volume of solution required to fill the chamber is about 6 micro-liters. Similarly, for epinephrine studies, the cells were assayed in HBSS without any BSA, and 1.6 micro-liters of 20 nM epinephrine solution was injected into the chamber to generate the epinephrine gradient.

The RWG biosensor exploits its evanescent wave to measure ligand-induced dynamic mass redistribution (DMR) signals in cells. The evanescent wave extends into the cells and exponentially decays over distance, forming a characteristic sensing distance of about 150-200 nm, implying that any optical response mediated through the receptor activation only represents an average over the portion of the cell that the evanescent wave is sampling. Such sampling with the biosensor is sufficient to differentiate the signaling of distinct classes of receptors in living cells under different stimulation conditions.

Like SPR, the RWG biosensor is sensitive to refractive index—an intrinsic property of biomolecules. Since the refractive index of a given volume within a cell is largely determined by the concentrations of bio-molecules such as proteins, we found, based on a three-layer waveguide grating theory, that a ligand-induced optical response is largely associated with dynamic mass redistribution. The relocation of cellular targets towards the sensor surface (e.g., relocation of intracellular targets to the activated receptors at the basal membrane surface) makes a positive contribution to the DMR (P-DMR); conversely, the movement of cellular targets away from the sensor surface (e.g., receptor internalization) is a negative contributor to the DMR (N-DMR). The aggregation of these events determines the kinetics and amplitudes of a ligand-induced DMR. However, recent studies, using plasmon waveguide resonance (PWR) technology and in vitro reconstituted G protein-coupled receptors immobilized onto the sensor surface showed that a ligand-induced optical response of the receptor-lipid membrane system consists of two components—changes in mass density and changes in structure. Since the RWG biosensor used here is unable to differentiate the contributions of these components, ligand-induced changes in organization of biomolecules in living cells may also contribute to the overall response measured.

For biosensor cellular assays, a baseline was established first. All studies were carried out at controlled temperature (22° C.) and with three replicates for each measurement, unless specifically mentioned.

### EXAMPLES

Some embodiments will be further clarified by the following non-limiting examples.

### Example 1

### The Diffusion-Induced Chemical Gradient Across the Biosensor

To simply visualize and measure the diffusion-based chemical gradient a small amount of chemical solution was injected into the chamber covering the biosensor, a 1.2 micro-liter solution containing 32 nano-molar fluorescently labeled EGF was injected from the inlet of the chamber. Fluorescence images across the whole chamber were recorded at many different time points. The fluorescence intensity across the chamber in a direction parallel to the line between the inlet and the outlet was recorded and analyzed as a function of location and time.

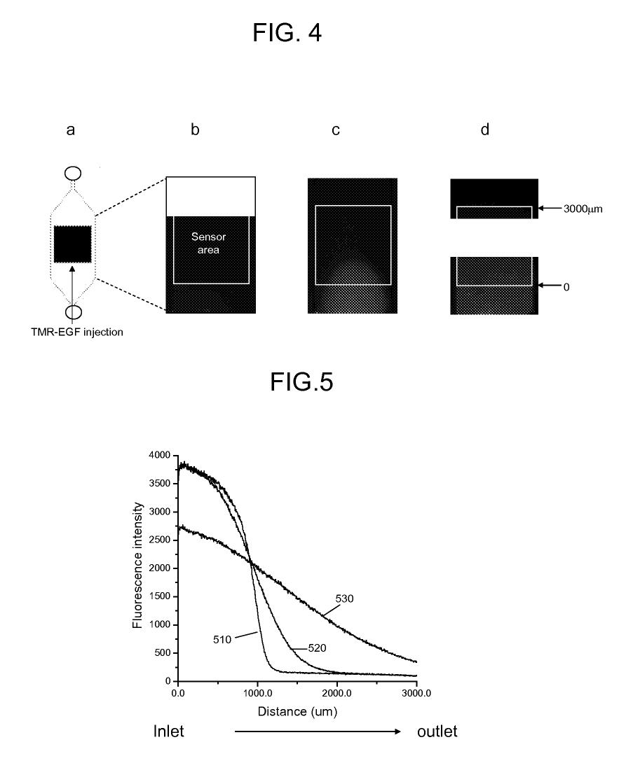

FIG. 4 show the fluorescence images of the solution across the chamber before (b) and at two different time points after the injection of 32 nM TMR-EGF: 10 sec (c) and 2 hours (d). The RWG biosensor within the chamber was about 3 mm×3 mm. A431 cells were cultured within the chamber until reaching high confluency (˜100%). FIG. 4a shows the orientation of the chamber as well as the injection direction of TMR-EGF solution. The images showed that there is no fluorescence before TMR-EGF addition (FIG. 4b), whereas the fluorescence is largely concentrated near the inlet side right after the TMR-EGF addition (FIG. 4c). In comparison, 2 hours after the TMR-EGF addition, the fluorescence can be detected at lower intensity and over a much bigger area (FIG. 4d), suggesting that the TMR-EGF diffused away from the original location right after the TMR-EGF addition. However, the TMR-EGF gradient is still maintained throughout the assay, as evidenced by the fluorescence intensity distribution across the chamber from the inlet to the outlet (FIG. 5). FIG. 5 shows the fluorescence intensity across the biosensor parallel to the direction from the inlet to the outlet of the chamber. The intensity profile 510 was obtained 10 sec after the TMR-EGF injection. The steepness of the concentration gradient indicates that not significant diffusion takes place within such short period of time. The intensity profiles 520 and 530 were obtained 10 min and 2 hours after the TMR-EGF injection, respectively. Although diffusion becomes obvious at these times, the concentration gradient of TMR-EGF across the biosensor was still maintained.

### Example 2

### The EGF Mediated Optical Responses of A431 Cells using the Global Stimulation Approach

Current cell assays generally measure cellular responses upon global stimulation with compounds. For example, simply pipetting an EGF solution into a well, exposes all the EGF receptors on the cell membrane to the same amount of EGF. These assays typically allow the compound to diffuse through the solution and the cultured cell layer, in order to stimulate receptors on all sides of the cell. As exampled in FIG. 2b, quiescent A431 cells responded with a typical DMR signal mediated through EGF receptors—an initial P-DMR, followed by a sequential N-DMR and a RP-DMR event. The cellular responses across the whole sensor area displayed small differences, primarily due to the unique cellular background at the single cell level and/or the heterogeneity of cell signaling at the single cell level.

### Example 3

### The EGF Gradient Mediated Optical Responses of A431 Cells at Different Locations

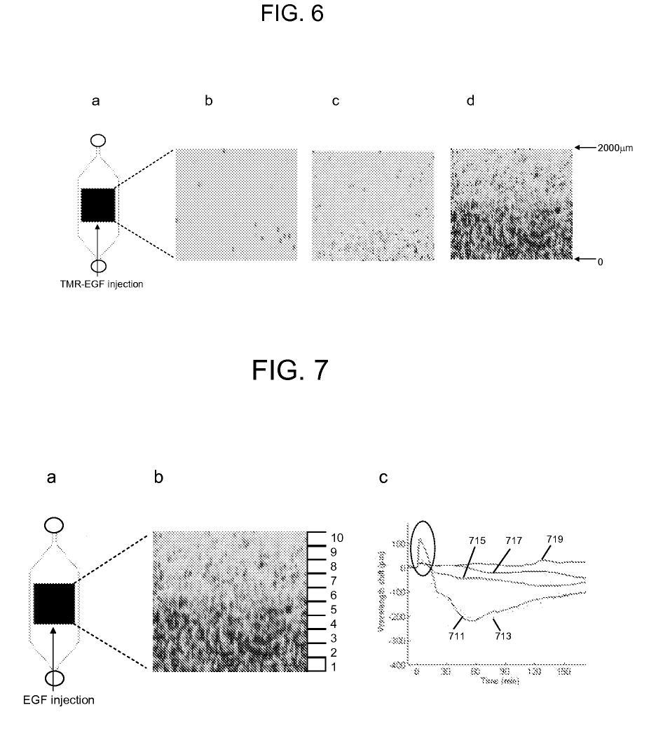

Using the simple injection approach, an EGF gradient was established (FIGS. 4 and 5). High resolution images of cellular responses using the imaging RWG biosensor system were recorded at different time points. As shown in FIGS. 6b, c and d, there are strong time- and location-dependent cellular responses. Before EGF addition, the resonant wavelength at each location was normalized, leading to a uniform baseline image (FIG. 6b). 10 sec after EGF addition, the bottom portion of the image, reflecting the cells near the inlet of the chamber, gave rise to an increased signal (FIG. 6c). However, 2 hrs after EGF addition, the bottom portion of the image, which is much larger than the area in FIG. 6c, gave rise to a decreased signal (FIG. 6d). Here the biosensor dimension was about 3 mm×3 mm. The cells reached high confluency and quiescent state through starvation at the time of assay.

Because of the high resolution of these label-free resonant images, cellular responses at different locations can be analyzed in many different ways. One of these uses is to study cellular responses at each pixel. Statistical analysis is then used to study cellular responses at the single cell level. These single cell responses are then linked to the location within the sensor, which is correlated with the chemical gradient that each cell at a specific location is encountered. Alternatively, the biosensor can be divided into many segments, and an averaged response of each segment can be generated. Such approach leads to cellular responses with minimal or little background noise, since the heterogeneity of cellular responses at single cell level is averaged out. FIG. 7 shows one example. A RWG biosensor having a dimension of 3 mm×3 mm was used. A431 cells were directly cultured within the chamber until high confluency, as examined using light microscopic imaging (data not show). 1.2 micro-liter 32 nM EGF solution was injected into the chamber from the inlet using a micropump (FIG. 7a). An example of resonant image of the biosensor having quiescent A431 cells was shown in FIG. 7b, which was obtained 2 hrs after the EGF injection. The image represented 380 pixel×380 pixel, in both x- and y-axis. Relative to the x-axis, the central half of the biosensor was used for further analysis. Then the central half was divided into 10 segments, as indicated in FIG. 7b. The averaged response of each segment was generated, and plotted as a function of time (FIG. 7c). Results showed that the cells located at the segment 1 led to a typical response (711), similar to those obtained using the global stimulation approach. The cells located at the segment 3 led to a truncated response (713)—it lacks the initial P-DMR event, suggesting no recruitment of intracellular proteins to the receptors located at the basal membrane of the cells. The cells located at the segment 5 and 7 also led to a truncated response but with much smaller amplitude (715, and 717, respectively). The cells near the outlet did not respond to the EGF gradient (719). Initially, the injected EGF solution only reached to segments 1 and 2. The cells located at these areas encountered a global stimulation by EGF. The cells located at near the edge of the injected EGF solution (i.e., the segment 3, 4, 5, and 6) encountered an EGF gradient, but with a different steepness. The closer to the segment 2, the more dramatic the EGF gradient is. Since EGF is a chemoattractant, A431 cells at the segments of 3 to 6 gave rise to a directional sensing, leading to a truncated optical response. These results suggest that the label-free biosensor cell assays, in conjunction with the chemical gradient generated can be used to study the early events of cell migration.

### Example 4

### The Epinephrine Gradient Mediated Optical Responses of A431 Cells at Different Locations

A chemoattractant may be an inorganic or organic substances possessing positive chemotaxis inducer effect in motile cells. The effect of chemoattractants are elicited via described or hypothetic chemotaxis receptors, including receptors such as tyrosine kinases such as EGFRs, and G protein-coupled receptors. The chemoattractant ability of a ligand may be target cell specific and concentration dependent. Many frequently investigated chemoattractants are formyl peptides and chemokines. Chemorepellents are substances that express an adverse effect on cell migration.

EGF is a chemoattractant, and can lead to the migration of A431 cells—a skin cancer cell line—acting through EGF receptors. Because of that, under the EGF gradient conditions, cells at different locations of the sensor surface gave rise to location-dependent cellular responses, which are atypical as compared to classical dose-dependent responses (FIGS. 6 and 7). In a classical dose-dependent response, the optical responses tend to exhibit dose-dependent changes in all kinetic features, rather than the truncation of signals observed under the gradient condition. Accordingly, we hypothesized that the occurrence of a truncated optical response of cells located at the area having the greatest chemical gradient can be used as an indicator to determine that the chemical is a chemoattractant.

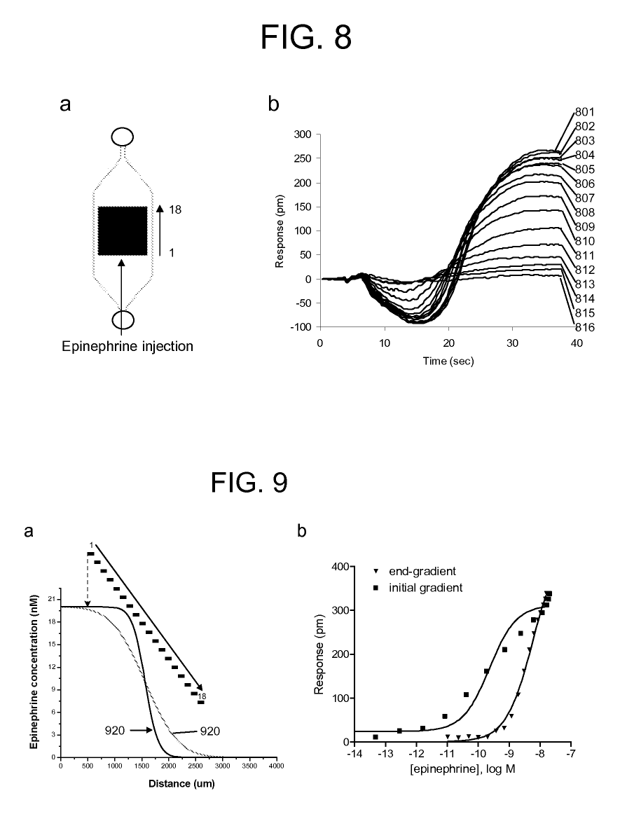

To test such an hypothesis, non-chemoattractant chemicals have to be examined, and are expected to trigger a location dependent response under the gradient condition that should be resemble a classical dose-dependent response. To do so, epinephrine—an agonist for endogenously expressed beta2-adrenergic receptors in A431—was used. A chamber having a RWG biosensor that is similar to the one used in FIGS. 6 and 7 was used. A431 cells were directly cultured within the chamber, and reached high confluency (100%). After starvation with a serum-free medium, the assay buffer (1×HBSS) was used to replace the serum free medium. Afterwards, 1.6 microliter 20 nM epinephrine was directly injected into the chamber. The RWG imaging system was used to record high resolution label-free cell responses across the whole sensor. Similar to the approach used in FIG. 7, the central portion of the biosensor was divided into 18 segments (FIG. 8a); each segment represents 100 pixel in x-axis×20 pixel in y-axis (meaning an area of 600 micron×120 micron). The averaged cellular responses of all segments were generated, and plotted as a function of time. FIG. 8 shows the location dependent kinetic responses of A431 cells, upon the epinephrine gradient. As expected, the epinephrine gradient generated led to a location-dependent cellular responses (FIG. 8b). The closer to the inlet area, the greater the cell response is. However, the cellular responses across the biosensor from the inlet to the outlet area closely resembled classical dose-dependent responses. This is in dramatic contrast with the EGF case (FIGS. 6 and 7), indicating that epinephrine, as expected, is not a chemoattractant.

To model the evolution of diffusion-driven concentration profiles in the present system, the solution for transient one-dimensional diffusion was used along with the accompanying initial and boundary conditions:

$$at t = 0 C = 0 for all x > 0 \\ at x = oo \frac{\partial C}{\partial x} = 0 for all t \geq 0 \qquad ( 2 )$$

Wherein C is the concentration of the chemical, D the diffusion coefficient. For epinephrine, its diffusion coefficient is about 6.85×10⁻¹⁰ m²/s. Based on the light microscopic image, the epinephrine solution injected into the chamber just reached and cover the segment 6, as indicated in FIG. 9a. Thus, the concentrations at each segment can be calculated, based on the equation (2). The cellular responses at each segment can be calculated, based on the amplitude of the P-DMR event. A dose-dependent curve can be extracted, and plot as a function of the initial (910) and end-point (920) gradient. The EC₅₀ of epinephrine can be calculated and found to be 0.23 nM, and 5.9 nM, when the initial and end-point gradient was used, respectively. The EC₅₀s obtained were close to that reported in literature, as well as those obtained using the global stimulation label-free cell assays (Fang, Y., and Ferric, A. M., FEBS Lett. 2008, Vol. 582, pp. 558-564). These results suggest that for non-chemoattractant chemicals, the present invention allows the estimation of the potency and efficacy of a chemical in a single assay.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

What is claimed is:

1. A method for studying cell motility, comprising the steps of:
providing a device, the device including:
a biosensor; and
a chamber having at least one chamber inlet for admission of at least one solution, a top and a bottom, wherein the bottom of the chamber includes or is adjacent to the biosensor;
attaching cells to the bottom of the chamber wherein the cells are in communication with the biosensor;
introducing at least one solution, the solution including at least one compound, into the chamber through the chamber inlet such that there is a compound gradient formed across the chamber; and
monitoring the biosensor to detect changes in the cells in response to the compound gradient.

2. The method according to claim 1, wherein the chamber further includes at least one chamber outlet.

3. The method according to claim 1, further including the steps of:
collecting a first set of data at a first time point from the biosensor at, at least two distinct areas on the biosensor;
gathering a second set of data at a second time point from the biosensor at, at least two distinct areas on the biosensor; and
comparing the first and the second data sets for each location, and the cellular profiles at, at least two distinct areas on the biosensor.

4. The method according to claim 1, wherein the device further includes:
a reservoir the reservoir having a reservoir outlet, wherein the reservoir stores solution before it is introduced into the chamber and the reservoir outlet is connected to the chamber inlet.

5. The method according to claim 4, wherein the device further includes:
a valve the valve having a valve inlet and a valve outlet wherein the valve inlet is connected to the reservoir outlet and the valve inlet and the valve outlet is connected to the chamber inlet.

6. The method according to claim 4, wherein the device further includes:
a pump the pump having a pump inlet and a pump outlet wherein the pump inlet is connected to the reservoir outlet and the pump outlet is connected to the chamber inlet.

7. The method according to claim 1, wherein the chamber further includes: a first inlet for the introduction of a first solution into the chamber; and a second inlet for the introduction of a second solution into the chamber, the first and the second solutions having different compositions, and
flowing the first and the second solutions across into the chamber such that a gradient is formed in the direction perpendicular to the flow across the cells on the bottom of the chamber.

8. The method according to claim 1, wherein the solution is introduced into the chamber through the chamber inlet at a controlled flow rate and duration.

9. The method according to claim 1, wherein the solution includes at least one compound selected from the group consisting of: proteins, nucleic acids, fatty acids, cells, viruses, small molecules, buffers, salts, and detergents.

10. The method according to claim 1, wherein the device further includes:
a temperature controller, wherein the controller regulates the temperature of the solution introduced into the chamber inlet.

11. The method according to claim 1, wherein the biosensor is a surface plasmon resonance sensor.

12. The method according to claim 1, wherein the biosensor is a resonant waveguide grating sensor.

13. The method according to claim 1, wherein the biosensor is an interferometer sensor.

14. A method for studying cell motility, comprising the steps of:
providing a device, the device including:
a biosensor; and
a chamber having at least one chamber inlet for admission of at least one solution, a top and a bottom, wherein the bottom of the chamber includes or is adjacent to the biosensor;
attaching cells to the bottom of the chamber wherein the cells are in communication with the biosensor;
introducing at least one solution, the solution including at least one compound, into the chamber through the chamber inlet such that there is a compound gradient formed across the chamber;
monitoring through the biosensor and at least two locations, a cellular response imparted by the compound; and
comparing the cellular responses from the at least two locations.

15. A device for monitoring cell migration, comprising:
a biosensor; and
a chamber the chamber having at least one chamber inlet for admission of at least one solution into the chamber so as to form a gradient in the chamber, a top and a bottom, wherein the distance between the top and the bottom of the chamber is in the range of about 200 microns to about 2 mm, and wherein the bottom of the chamber includes or is adjacent to the biosensor and includes at least one cell attached to the bottom of the chamber.

16. The device according to claim 15, wherein the distance between the top and the bottom of the chamber is in the range of about 300 microns to about 1 mm.

17. The device according to claim 15, wherein the biosensor is selected from the group consisting of: a surface plasmon resonance sensor, a resonant waveguide grating sensor and an interferometer sensor.

18. The device according to claim 15, wherein the device further includes: at least one outlet.

19. The device according to claim 15, further including a temperature regulator, wherein the regulator regulates the temperature of the solution introducing into the chamber.

20. The device according to claim 15, further including:
a reservoir for storing solution before it is introduced into the chamber, the reservoir having a reservoir outlet;
a valve the valve having a valve inlet and a valve outlet, wherein the valve inlet is connected to the reservoir outlet and valve outlet is connected to the chamber inlet.
